(19) United States
(12) Patent Application Publication
(10) Pub. No.: US 2005/0021238 A1
(43) Pub. Date: Jan. 27, 2005

(54) Method and system for chromosome correction in genetic optimazation process

(76) Inventors: Tyson McGuffin, Fort Collins, CO (US); Thomas W. Chen, Fort Collins, CO (US); Dave Anderson, Fort Collins, CO (US); David C. Burden, Fort Collins, CO (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/624,266

(22) Filed: Jul. 21, 2003

(51) Int. Cl. G06F 19/00; G01N 33/48; G01N 33/50

(52) U.S. Cl. 702/20

(57) ABSTRACT

The convergence speed of a computer-implemented genetic optimization process is improved through the correction of child chromosomes containing undesirable gene combinations. Undesirable gene combinations may be identified through application of heuristic techniques, statistical techniques, or a combination of the two.

### FIELD OF THE INVENTION

The present invention relates generally to computerized optimization techniques and more specifically to techniques based on a genetic model.

### BACKGROUND OF THE INVENTION

Due to the large number of variables involved, some optimization problems have an exceedingly large solution space. For example, selecting design parameters such as size for each of thousands of instances (occurrences of cells) in an integrated circuit to minimize power consumption while maintaining acceptable timing performance is such a problem.

One approach to solving complex optimization problems is genetic optimization, an iterative, computer-implemented technique in which candidate solutions are generated using a genetic model. The genetic model typically includes a set of N randomly generated chromosomes, each chromosome comprising a number of genes. Since each gene represents a particular state of a parameter to be optimized in one part of a system (e.g., the size of one instance in an integrated circuit), each chromosome represents a possible solution to the global optimization problem. In a typical application, the configuration specified by each chromosome is evaluated, and a fitness score is assigned to each chromosome. For example, in the case of an integrated circuit design, circuits having the characteristics of each respective chromosome may be simulated to assign a fitness score to each based on its power consumption and timing performance. Once each chromosome has been assigned a score, the N chromosomes may be “mated” or “mutated” in various ways to create other potential solutions or “children,” which may, in turn, be evaluated and assigned a fitness score. Each pass through this reproduction procedure may be termed a “generation” of the genetic optimization process. If a child has a higher score than one of the original N chromosomes, the chromosome with the lowest score may be discarded. Eventually, the candidate solutions generated in this fashion may converge to the global optimum.

A difficulty often arises in genetic optimization, however, that a child chromosome may have a low fitness score due to the presence of just a few undesirable genes. In some cases, such an undesirable gene combination would be obvious to a circuit designer. For example, the size of one or more instances in the context of a particular circuit may clearly lie outside the acceptable range. Consequently, the genetic optimization process may proceed through many generations of trial and error before the obviously undesirable gene combination is finally purged from the chromosome pool.

It is thus apparent that there is a need in the art for an improved method and system for finding a solution to an optimization problem using a genetic optimization process.

### SUMMARY OF THE INVENTION

A method is provided for correcting chromosomes containing undesirable gene combinations in a computer-implemented optimization process based on a genetic model. A system and a computer-readable storage medium for implementing the method are also provided.

Other aspects and advantages of the present invention will become apparent from the following detailed description, taken in conjunction with the accompanying drawings, illustrating by way of example the principles of the invention.

### BRIEF DESCRIPTION OF THE DRAWINGS

FIGS. 1A-1C are illustrations showing how instances in an integrated circuit may be mapped onto a genetic algorithm in accordance with an illustrative embodiment of the invention.

FIG. 2 is a conceptual diagram of chromosome correction in a genetic optimization process in accordance with an illustrative embodiment of the invention.

FIG. 3 is a flowchart of a genetic optimization process in accordance with an illustrative embodiment of the invention.

FIGS. 4A and 4B are illustrations of a specific example of chromosome correction in accordance with an illustrative embodiment of the invention.

FIG. 5A is an illustration of a system for carrying out the methods of the invention in accordance with an illustrative embodiment of the invention.

FIG. 5B is an illustration of networked processing nodes for carrying out the methods of the invention in accordance with an illustrative embodiment of the invention.

### DETAILED DESCRIPTION OF THE INVENTION

The speed of convergence of a genetic optimization process may be improved by detecting the presence of undesirable gene combinations in the child chromosomes produced during each generation of the process. Once an attempt has been made to correct the undesirable gene combinations, the fitness score of each putatively corrected child chromosome may be updated. If necessary, the attempted correction and re-evaluation of fitness may be repeated in an iterative fashion. When the chromosome pool is subsequently updated for the next generation, the corrected child chromosomes and their associated updated fitness scores may be included in the selection process. Since undesirable gene combinations may be eliminated during the chromosome evaluation step, the genetic optimization process may converge to a solution much more rapidly than in a purely trial-and-error approach.

The balance of this detailed description provides additional details about the principles of the invention through the discussion of illustrative embodiments thereof. Although the illustrative embodiments presented concern integrated circuit design, the principles of the invention may be applied to any genetic optimization process.

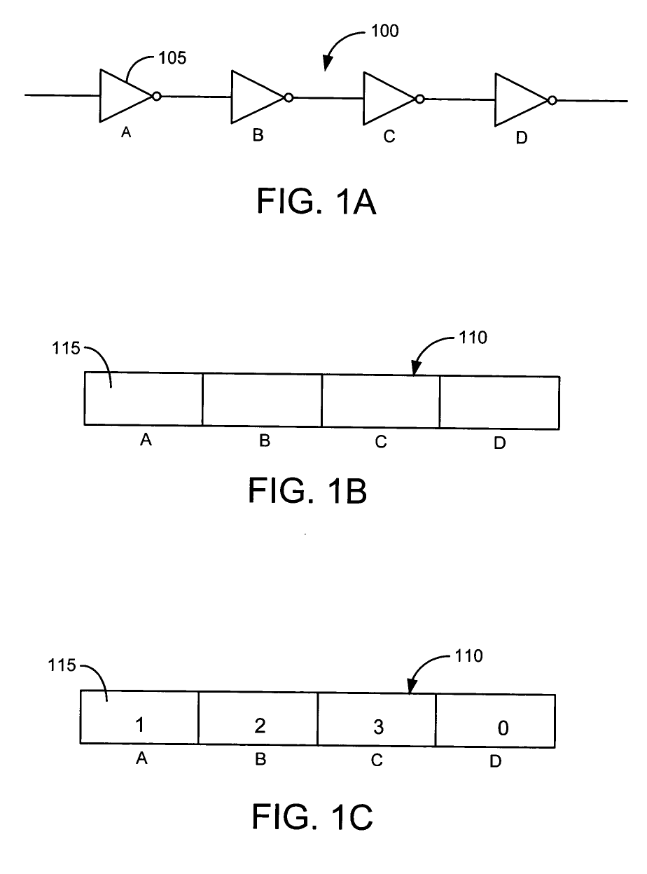

FIGS. 1A-1C illustrate how instances in an integrated circuit may be mapped onto a genetic optimization process in accordance with an illustrative embodiment of the invention. FIG. 1A is a gate-level diagram of a simple integrated circuit 100. In this simplified example, integrated circuit 100 comprises four instances (replications of a cell) 105, each of which is an inverter. Each instance 105 is labeled A-D to facilitate further description. FIG. 1B is a conceptual diagram of a chromosome 110 comprising a gene 115 for each instance 105 for a total of four genes. For example, each gene 115 may indicate the size of its corresponding instance 105. Genes 115 may be represented conveniently as integers. For example, a gene 115 representing the size of its corresponding instance may comprise an integer between 0 and 5, inclusive, corresponding to a set of predetermined physical sizes. An example of a particular chromosome conforming to the foregoing scheme is shown in FIG. 1C. In other embodiments, multiple genes 115 corresponding to various circuit design parameters may be associated with each instance 105 in chromosome 110.

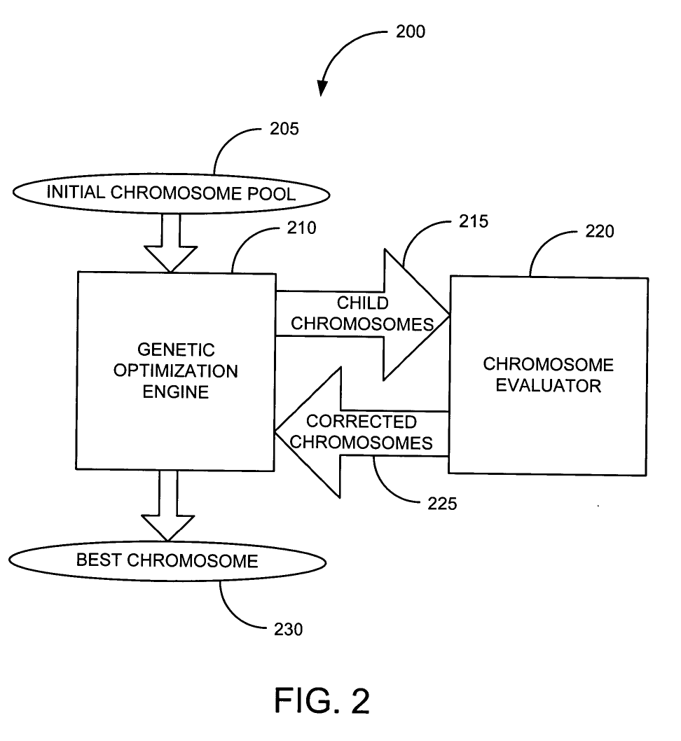

FIG. 2 is a conceptual diagram of chromosome correction in a genetic optimization process 200 in accordance with an illustrative embodiment of the invention. Genetic optimization process 200 begins with an initial (randomly generated) chromosome pool 205. Genetic optimization engine 210 generates, through a reproduction procedure, a set of child chromosomes 215 during each generation of genetic optimization process 200. Reproduction methods are discussed in greater detail in jointly owned U.S. patent application Ser. No. 10/057,245, “Method and System for Reproduction in a Genetic Optimization Process,” which was filed on Jan. 25, 2002. By simulating the performance of integrated circuits 100 having design characteristics corresponding to each of the respective child chromosomes 215, a fitness score may be assigned to each child chromosome 215. In integrated circuit design, the objective is typically to achieve an acceptable trade-off between power consumption and speed. Chromosome evaluator 220 may examine each child chromosome for undesirable gene combinations marring an otherwise acceptable solution to the optimization problem. More will be said about how undesirable gene combinations may be recognized in a later portion of this detailed description. Chromosome evaluator 220 may produce a set of putatively corrected child chromosomes, whose fitness scores may be updated accordingly. If necessary, the correction and re-evaluation (assigning of updated fitness scores) steps may be repeated. In one embodiment, a “greedy” optimization may be applied to the faulty gene combinations. The corrected child chromosomes 225 and their associated updated fitness scores produced by chromosome evaluator 220 may be subsequently fed to genetic optimization engine 210 for consideration in the updating of chromosome pool 205 for the next generation. Eventually, the genetic optimization converges on a best chromosome 230.

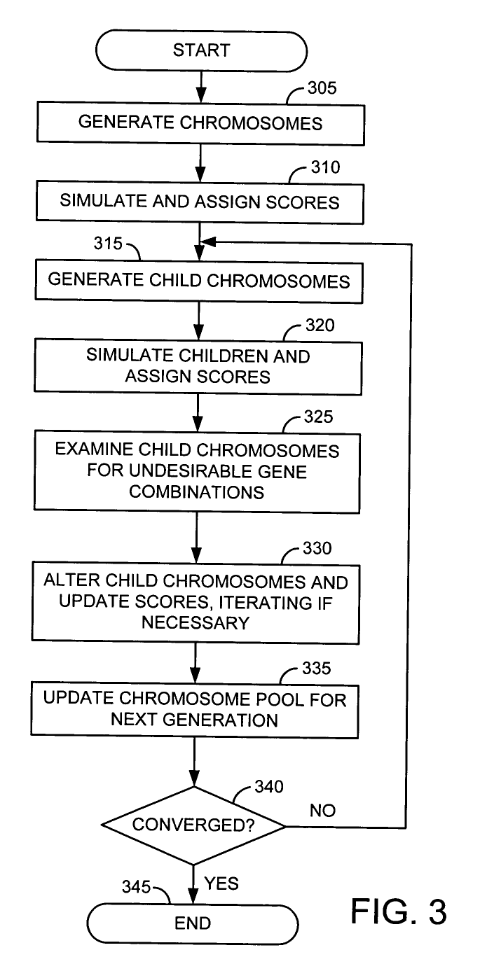

FIG. 3 is a flowchart of a genetic optimization process 200 in accordance with an illustrative embodiment of the invention. At 305, a set of N unique chromosomes 110 may be generated. For example, a random number generator may be used to assign a value to each gene 115 in each of the N chromosomes 110. The set of N chromosomes 110 will also be referred to as a “chromosome pool” 205 in this description. In practice, the values generated by the random number generator may be pseudorandom. The performance of integrated circuits 100 having design characteristics corresponding to each of the respective N chromosomes 110 may be simulated at 310. In this illustrative embodiment, the power consumption of integrated circuit 100 is to be minimized while maintaining acceptable timing. At 310, a score may be assigned to each chromosome indicating how well it meets the design goals. For example, the score may be a number proportional to the total power consumption of the integrated circuit 100. At 315, a set of child chromosomes 215 is generated through a reproduction process (e.g., mating and/or mutation). At 320, integrated circuits 100 corresponding to child chromosomes 215 are simulated, and a fitness score is assigned to each child chromosome 215.

At 325, the child chromosomes 215 may be examined for the presence of undesirable gene combinations. There are at least two broad categories of approaches to identifying undesirable gene combinations in child chromosomes 215 at 325: heuristic and statistical. In some embodiments, these two approaches may be combined.

In the heuristic approach, chromosome evaluator 220 applies programmed a priori knowledge of the constraints governing a particular optimization problem to recognize undesirable gene combinations in child chromosomes 215. For example, an integrated circuit designer may know that a particular gene combination (e.g., a combination of instance sizes) will not produce acceptable timing performance in an integrated circuit 100. Such intelligence may be programmed into chromosome evaluator 220. In the heuristic approach, side information about the specific optimization problem being solved is thus applied to recognize child chromosomes 215 containing undesirable gene combinations.

In the statistical approach, chromosome evaluator 220 may employ one or more statistical techniques to identify undesirable gene combinations in child chromosomes 215. Many different statistical techniques may be used, all of which are considered to be within the scope of the invention as claimed. One example is the use of neural networks in conjunction with the overall genetic optimization process 200. A neural network may be trained to recognize “good” and “bad” gene combinations (subsets of genes 115) within a child chromosome 215 through many repeated trials in which the neural network is informed whether each gene combination in the training set is “good” or “bad,” according to the fitness score associated with the child chromosome 215. When the neural network is then presented with a gene combination it has never encountered, it may recognize the gene combination as desirable or undesirable based on its cumulative “experience” acquired during the training phase. Of course, the neural network does not “know” why a particular gene combination is desirable or undesirable as a circuit designer would in programming chromosome evaluator 220 for the heuristic approach. Thus, the gene corrections may need to be pseudo-random as opposed to the “greedy” correction employed in a heuristic approach. Those skilled in the art will recognize that the classification of a gene combination need not be binary (“good” vs. “bad”) as indicated above, but the binary case is considered here for simplicity.

One illustrative neural network implementation is as follows. A list of paths in integrated circuit 100 may be analyzed to identify critical paths. A critical path is one that has timing problems or that barely meets timing requirements. Since a critical path is rarely isolated from all other critical paths, critical paths sharing a substantial portion of gates (e.g., 50 percent) may be grouped together and assigned to the same neural network (one neural network per group of critical paths). If possible, every critical path is included in a group, and each gate through each critical path within a group is an input to the neural network associated with that group. A neural network may be trained on each subset of genes associated with the corresponding critical paths. That is, each neural network may be trained to identify desirable and undesirable gene combinations for a particular subset of genes corresponding to instances 105 in integrated circuit 100.

The undesirable gene combinations may be altered at 330 in an attempt to correct them. Also, the fitness score associated with each putatively corrected child chromosome 215 may be updated to determine whether the alteration was indeed a correction of the faulty gene combination. This may be accomplished by incrementally re-simulating the corresponding integrated circuit 100 (i.e., simulating only the necessary paths and instances affected by the attempted correction) or by wholesale re-simulation of the entire integrated circuit 100. Those skilled in the art will immediately recognize the speed and efficiency advantages of the former approach.

Alterations of undesirable gene patterns at 330 may be made in at least four ways, which may be combined in some embodiments. First, the faulty genes may be randomly altered. For example, the size of an instance 105 may be bumped up or down randomly. Second, the faulty genes may be altered deterministically based on a priori knowledge of the constraints present in a particular optimization problem. For example, chromosome evaluator 220 may be programmed with certain “rules of thumb” regarding the minimum acceptable size of instances 105 in particular situations. For example, the fan-out of instance 105 may necessitate a particular minimum size. Such knowledge may be applied directly to correct obviously undesirable gene combinations without the need to wait for them to be eliminated through trial-and-error. Such an approach is consistent with the heuristic approach to identifying undesirable gene combinations described above. Third, undesirable gene combinations may be corrected using a “greedy” optimization. In this approach, a parameter such as size is adjusted up or down for a particular instance 105, and if the associated fitness score improves, further adjustment in the same sense is made in the hope that the fitness score will improve even more. This process is repeated until the fitness score again worsens, at which point the closest local minimum will have been found. Finally, in conjunction with a neural-network statistical approach to identifying undesirable gene combinations, all neural networks may be consulted to find the one that is the most dissatisfied (i.e., its figure of merit is the lowest among the neural networks). Using a directed random approach, the inputs (combination of genes) to that neural network may be tweaked until its figure of merit improves. The corrected child chromosome 215 may then be re-evaluated to obtain its updated fitness score. Further, the neural network's training may be reinforced if the correction turns out to be favorable upon re-evaluation (i.e., re-simulation of integrated circuit 100).

The best N chromosomes from among the original N and the child chromosomes may be retained for the next generation at 335. That is, the chromosome pool 205 is updated for the next generation at 335. If the N chromosomes have converged to within a predetermined tolerance at 340, the process may be terminated at 345. Otherwise, control may return to 315, and a new generation may begin based upon the updated chromosome pool 205.

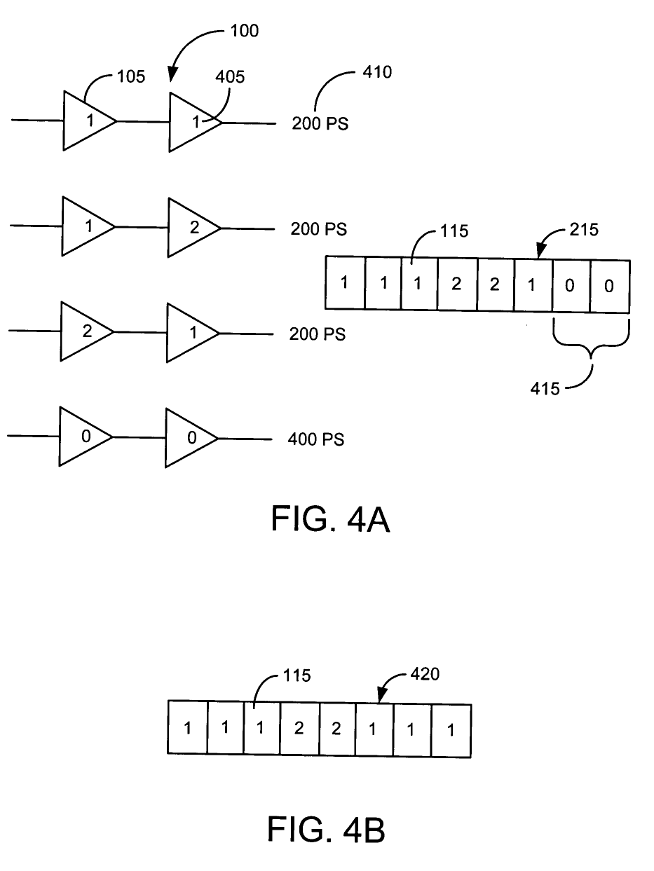

FIGS. 4A and 4B are illustrations of a specific example of chromosome correction in accordance with an illustrative embodiment of the invention. In FIG. 4A, integrated circuit 100 comprises four parallel paths, each having two instances 105. Each instance 105 is labeled with an index 405 indicating its size. Also, each parallel path is labeled with its propagation time 410 in picoseconds (ps). Note that the fourth parallel path has a propagation time 410 that is longer than the other three (400 ps). A child chromosome 215 comprising eight genes 115 corresponding to integrated circuit 100 is also shown. In this simplified example, each gene 115 of child chromosome 215 contains the size index 405 of the corresponding instance 105. In child chromosome 215, gene 115 of each of the two instances 105 in the fourth parallel path is zero (smaller than that of the other instances 105). This may be referred to as an undesirable gene combination 415. In this simplified example, a circuit designer examining child chromosome 215 would recognize undesirable gene combination 415. Genetic optimization process 200 may take many more generations, using only conventional random mating or mutation algorithms, to correct this obvious flaw in child chromosome 215. Instead, a priori knowledge of such constraints on integrated circuit 100 may be programmed into chromosome evaluator 220, and a suitable alteration may be made to child chromosome 215 to correct undesirable gene combination 415 and thereby speed up convergence.

FIG. 4B illustrates an altered child chromosome 420, in which the genes 115 in undesirable gene combination 415 have been increased from zero to one. At this point, chromosome evaluator 220 may re-simulate the timing performance of integrated circuit 100 with the adjusted sizes for the last two instances 105 to confirm that the alteration is indeed an improvement (a corrected child chromosome 225). As mentioned above, this re-simulation to assign an updated fitness score may be performed incrementally. That is, only the fourth path involving the last two instances 105 need be re-simulated. Alternatively, the entire integrated circuit 100 may be re-simulated according to altered child chromosome 420 at the expense of decreased speed and efficiency.

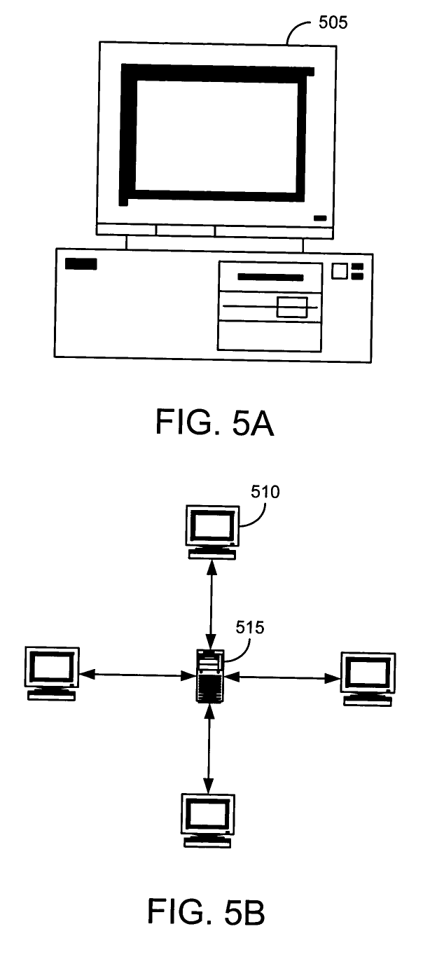

FIG. 5A illustrates a system for carrying out the methods of the invention, in accordance with an illustrative embodiment of the invention. FIG. 5A depicts a general purpose computer 505. The methods of the invention may be programmed to execute on such a general purpose computer 505 or, alternatively, the invention may be implemented in a special-purpose (e.g., high-performance) computer, in custom hardware, or in any combination of hardware and software.

FIG. 5B illustrates networked processing nodes for carrying out the methods of the invention in accordance with an illustrative embodiment of the invention. In this particular example, a plurality of workstations 510 are networked together via server 515. It is advantageous, for example, to send each child chromosome 215 to a different processing node 510 in parallel fashion to perform the simulation of the corresponding integrated circuit 100 and assign the associated fitness score.

Program code implementing the invention may also be stored on a computer-readable storage medium. Examples of computer-readable storage media include solid-state read-only memory, magnetic disks, and optical disks. In such an implementation, the contents of the computer-readable storage medium may comprise a first code segment configured to generate a set of child chromosomes 215 during each generation, a second code segment configured to examine the child chromosomes 215 for undesirable gene combinations 415, a third code segment configured to alter one or more undesirable gene combinations 415, and a fourth code segment configured to evaluate the fitness of the putatively corrected child chromosomes 420 before chromosome pool 205 is updated for the next generation.

The foregoing description of the present invention has been presented for the purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

1. A method for searching for an optimal solution to an optimization problem using a computer-implemented process based on a genetic model, comprising:
generating, during each of a series of generations of the computer-implemented process, a set of child chromosomes, each child chromosome comprising at least one gene;
examining the child chromosomes for undesirable gene combinations;
altering the undesirable gene combinations to produce a set of putatively corrected child chromosomes; and
evaluating the fitness as the optimal solution of each of the putatively corrected child chromosomes prior to updating a chromosome pool for use in the successive generation.

2. The method of claim 1, wherein the undesirable gene combinations are identified based on a priori knowledge of constraints on the optimization problem.

3. The method of claim 1, wherein the undesirable gene combinations are identified by use of a statistical technique.

4. The method of claim 3, wherein the statistical technique comprises training a neural network on at least one gene subset within the child chromosomes.

5. The method of claim 1, wherein the undesirable gene combinations are identified based on a combination of a priori knowledge of constraints on the optimization problem and the use of a statistical technique.

6. The method of claim 1, wherein altering the undesirable gene combinations to produce a set of putatively corrected child chromosomes comprises deterministically altering at least one undesirable gene combination based on a priori knowledge of constraints on the optimization problem.

7. The method of claim 1, wherein altering the undesirable gene combinations to produce a set of putatively corrected child chromosomes comprises randomly altering at least one undesirable gene combination.

8. The method of claim 1, wherein altering the undesirable gene combinations to produce a set of putatively corrected child chromosomes comprises altering at least one undesirable gene combination in accordance with a greedy optimization.

9. The method of claim 1, wherein the optimization problem comprises optimizing at least one characteristic of an integrated circuit.

10. A method for searching for an optimal solution to an optimization problem using a computer-implemented process based on a genetic model, comprising:
representing candidates for the optimal solution as a chromosome pool, each chromosome in the chromosome pool comprising at least one gene; and
performing the following steps iteratively during each of a series of generations until a chromosome is determined to be the optimal solution to the optimization problem:
generating, through a reproduction process, a set of child chromosomes,
assigning a fitness score to each child chromosome,
examining the child chromosomes for undesirable gene combinations,
altering the undesirable gene combinations to produce a set of putatively corrected child chromosomes,
assigning an updated fitness score to each putatively corrected child chromosome, and
updating the chromosome pool for the successive generation.

11. The method of claim 10, wherein the undesirable gene combinations are identified based on a priori knowledge of constraints on the optimization problem.

12. The method of claim 10, wherein the undesirable gene combinations are identified by use of a statistical technique.

13. The method of claim 12, wherein the statistical technique comprises training a neural network on at least one gene subset within the child chromosomes.

14. The method of claim 10, wherein the undesirable gene combinations are identified based on a combination of priori knowledge of constraints on the optimization problem and the use of a statistical technique.

15. The method of claim 10, wherein altering the undesirable gene combinations to produce a set of putatively corrected child chromosomes comprises deterministically altering at least one undesirable gene combination based on a priori knowledge of constraints on the optimization problem.

16. The method of claim 10, wherein altering the undesirable gene combinations to produce a set of putatively corrected child chromosomes comprises randomly altering at least one undesirable gene combination.

17. The method of claim 10, wherein altering the undesirable gene combinations to produce a set of putatively corrected child chromosomes comprises altering at least one undesirable gene combination in accordance with a greedy optimization.

18. The method of claim 10, wherein the optimization problem comprises optimizing at least one characteristic of an integrated circuit.

19. A system programmed to perform the following method:
generating, during each of a series of generations of a computer-implemented process based on a genetic model for solving an optimization problem, a set of child chromosomes, each child chromosome comprising at least one gene;
examining the child chromosomes for undesirable gene combinations;
altering the undesirable gene combinations to produce a set of putatively corrected child chromosomes; and
evaluating the fitness as a solution to the optimization problem of each of the putatively corrected child chromosomes prior to updating a chromosome pool for use in the successive generation.

20. The system of claim 19, wherein the system comprises a plurality of networked processing nodes.

21. A system programmed to perform the following method:
representing candidates for an optimal solution to an optimization problem as a chromosome pool, each chromosome in the chromosome pool comprising at least one gene; and
performing the following steps iteratively during each of a series of generations of a process based on a genetic model until a chromosome is determined to be the optimal solution to the optimization problem:
generating, through a reproduction process, a set of child chromosomes,
assigning a fitness score to each child chromosome,
examining the child chromosomes for undesirable gene combinations,
altering the undesirable gene combinations to produce a set of putatively corrected child chromosomes,
assigning an updated fitness score to each putatively corrected child chromosome, and
updating the chromosome pool for the successive generation.

22. The system of claim 21, wherein the system comprises a plurality of networked processing nodes.

23. A system for searching for an optimal solution to an optimization problem using a computer-implemented process based on a genetic model, comprising:
means for generating, during each of a series of generations of the computer-implemented process, a set of child chromosomes, each child chromosome comprising at least one gene;
means for examining the child chromosomes for undesirable gene combinations;
means for altering the undesirable gene combinations to produce a set of putatively corrected child chromosomes; and
means for evaluating the fitness as the optimal solution of each of the putatively corrected child chromosomes prior to updating a chromosome pool for use in the successive generation.

24. A computer-readable storage medium containing program code to solve an optimization problem according to a process based on a genetic paradigm, comprising:
a first code segment configured to generate, during each of a series of generations of the process, a set of child chromosomes, each child chromosome comprising at least one gene;
a second code segment configured to examine the child chromosomes for undesirable gene combinations;
a third code segment configured to alter the undesirable gene combinations to produce a set of putatively corrected child chromosomes; and
a fourth code segment configured to evaluate the fitness as a solution to the optimization problem of each of the putatively corrected child chromosomes prior to updating a chromosome pool for use in the successive generation.
